(19) United States
(12) Patent Application Publication
(10) Pub. No.: US 2010/0178257 A1
(43) Pub. Date: Jul. 15, 2010

(54) BLOCK POLYMER, COSMETIC COMPOSITION COMPRISING IT AND COSMETIC TREATMENT PROCESS

(75) Inventor: Celine FARCET, Les Pavillons Sous Bois (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'OREAL, Paris (FR)

(21) Appl. No.: 12/506,511

(22) Filed: Jul. 21, 2009

Related U.S. Application Data
(60) Provisional application No. 61/095,056, filed on Sep. 8, 2008.

(30) Foreign Application Priority Data
Jul. 24, 2008 (FR) 08 55069

(51) Int. Cl. A61K 8/90; A61Q 17/04; A61Q 3/00; A61Q 1/12; A61Q 1/06; A61Q 5/06; A61Q 5/00; C08L 53/00

(52) U.S. Cl. 424/59; 424/61; 424/63; 424/64; 424/70.7; 424/70.16; 424/78.03; 525/222

(57) ABSTRACT

A block polymer containing, in a block with a Tg of less than 20° C., at least one monomer of formula (I):

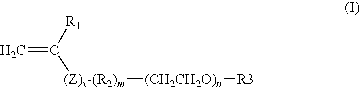

The invention also relates to a cosmetic composition containing the block polymer in a cosmetically acceptable medium, and also to a cosmetic process for treating keratin materials using the composition, and most particularly to a process for making up the lips.

### REFERENCE TO PRIOR APPLICATIONS

This application claims priority to U.S. provisional application Ser. No. 61/095,056, filed Sep. 8, 2008; and to French patent application 08 55069, filed Jul. 24, 2008, both incorporated herein by reference.

### FIELD OF THE INVENTION

The present invention relates to novel polymers, and to their use in cosmetics; the invention also relates to compositions, especially cosmetic compositions, comprising these polymers.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Various types of polymer are conventionally used in cosmetic compositions on account of the various properties that they can impart thereto. They are used, for example, in compositions for making up or caring for the skin, the lips or the integuments, such as nail varnishes or haircare compositions. However, by using within the same composition two polymers that are incompatible, i.e. that are immiscible in the same solvent, the formulator is confronted, due to the incompatibility of the polymers, with problems of phase separation, or even of decantation, and in general with the production of a non-homogeneous composition. Hitherto, these problems were able to be solved only by the presence in the composition of a compound for compatibilizing the polymers.

To overcome this problem, patent application EP 1 411 069 has proposed polymers of particular structure, comprising at least two mutually incompatible blocks connected together via an intermediate block that comprises at least one constituent monomer of each of the two blocks.

The patent application mainly describes block polymers prepared from monomers of alkyl, especially methyl, isobutyl, isobornyl or trifluoroethyl, acrylate and methacrylate or (meth)acrylic acid type. These polymers may generally be conveyed, and are especially soluble, in organic solvents such as short esters (butyl or ethyl acetate), short alcohols such as ethanol, or aliphatic alkanes such as isododecane. They enable the production of glossy and sparingly tacky films, which are particularly appreciated in certain cosmetic fields.

However, these films have a certain brittle nature and also sensitivity to fatty substances, especially over time.

Now, what are sought, especially in the field of makeup, are polymers that are capable of resisting and displaying fastness with respect to external attack, especially “attack” by fatty substances, for instance food oil or sebum.

Furthermore, these polymers do not enable optimum cosmetic properties to be obtained, especially in terms of glidance, absence of tack, comfort, staying power of the composition and feel.

The inventor has discovered, surprisingly, novel polymers, which may be conveyed in organic medium, and which make it possible to obtain good cosmetic properties such as good adhesion to the support (skin or hair) and thus good staying power of the cosmetic composition.

### DETAILED DESCRIPTION OF THE PREFERRED EMBODIMENTS

One subject of the present invention is a block polymer comprising at least one first block and at least one second block, which are mutually incompatible, in which one of the blocks has a glass transition temperature (Tg) of less than or equal to 20° C. and comprises 0.5% to 100% by weight, relative to the weight of the block, of at least one monomer of formula (I) as defined hereinbelow.

A subject of the invention is also the cosmetic composition comprising the polymer in a cosmetically acceptable medium.

The comfort of the cosmetic composition is improved, as are its feel, its softness, its glidance and its tack-free nature.

The composition makes it possible to obtain a film that is glossy and sparingly tacky, while at the same time also being sparingly brittle or non-brittle and sufficiently flexible, and advantageously having good resistance to fatty substances.

It may be thought that the presence of PEG units in the polymer simultaneously affords glidance and insensitivity to oils, i.e. an improvement in the comfort and staying power, and also volume and gloss.

The polymers bearing PEG units have increased comfort properties by virtue of the glidance and the lubrication of the PEG units that develops in a humid environment.

The compositions according to the invention may afford, in the field of makeup, increased comfort properties, especially improved glidance, in particular in a humid environment. Furthermore, they may show improved resistance to external attack (meals, sebum) and to rubbing. The comfort and staying power are thus improved.

In the more particular case of lipsticks, the use of saliva on the lips may make it possible to “relubricate” in order to prolong the comfort and staying power, and also to generate a swelling effect, or volume, which will give a pouty effect; the humid film thus created at the surface protects the deposit; the polymers according to the invention may thus afford volume by swelling in the presence of water, and also a “wet” gloss appearance.

Furthermore, the polymers may be conveyed in oily media that are particularly favoured in the field of makeup; now, polymers comprising PEG (polyethylene glycol) units were hitherto generally used in aqueous solution or dispersion, this medium being difficult to formulate in the field of makeup.

The composition according to the invention thus comprises at least one block polymer comprising at least one first block and at least one second block, which may have different glass transition temperatures (Tg) and which are advantageously mutually incompatible.

It is pointed out that the terms “first block” and “second block” do not in any way condition the order of the blocks in the structure of the polymer.

The expression “mutually incompatible blocks” means that the mixture formed from the polymer corresponding to the first block and the polymer corresponding to the second block is immiscible in the polymerization solvent present in weight majority for the block polymer, at room temperature (25° C.) and atmospheric pressure (10⁵ Pa), for a content of the polymer mixture of greater than or equal to 5% by weight, relative to the total weight of the mixture (polymers and solvent), it being understood that:

i) the polymers are present in the mixture in a content such that the respective weight ratio ranges from 10/90 to 90/10, and that

ii) each of the polymers corresponding to the first and second blocks has an average (weight-average or number-average) molecular mass equal to that of the block polymer±15%.

In the case of a mixture of polymerization solvents, should two or more solvents be present, the polymer mixture is immiscible in at least one of them. Needless to say, in the case of a polymerization performed in a single solvent, this solvent is the majority solvent.

The first and second blocks may be advantageously connected together via an intermediate segment comprising at least one constituent monomer ml of the first block and at least one constituent monomer m2 of the second block. Preferentially, the intermediate segment forms an intermediate block. Preferably, m2 is different from m1. The intermediate segment or block may especially allow these first and second blocks to be “compatibilized”.

The block polymer used in the context of the invention is advantageously a linear, branched or grafted, preferably linear, block ethylenic polymer, capable of forming a deposit, more particularly a film (film-forming).

The term “ethylenic polymer” refers to a polymer obtained by polymerization of monomers comprising an ethylenic unsaturation.

The term “block polymer” refers to a polymer comprising at least two different blocks and preferably at least three different blocks; preferably diblock or triblock polymers.

The polymer according to the invention is linear, branched or grafted; preferably, it does not comprise any multifunctional monomer or compound, added deliberately, which is capable of generating branches and/or crosslinks.

The term “polymer forming a deposit” refers to a polymer that is capable of forming, by itself or in the presence of an auxiliary, a deposit that adheres to a support, especially to keratin materials.

The term ‘film-forming polymer’ refers to a polymer that is capable of forming, by itself or in the presence of an auxiliary film-forming agent, a continuous film that adheres to a support, especially to keratin materials.

Each block of the polymer according to the invention is derived from one type of monomer or from several different types of monomer. This means that each block may be formed from a homopolymer or a copolymer, which may be statistical, alternating or the like.

Advantageously, when it is present, the intermediate segment or block comprising at least one constituent monomer of the first block and at least one constituent monomer of the second block of the polymer is a statistical polymer. Preferably, the intermediate segment or block is derived essentially from constituent monomers of the first block and of the second block. The term “essentially” means at least 85%, preferably at least 90%, better still 95% and even better still 100%.

According to the invention, the first and second blocks preferably have different glass transition temperatures, with a difference generally greater than 5° C., preferably greater than 10° C. and better still greater than 20° C. Advantageously, the intermediate block has a glass transition temperature Tg that is between the glass transition temperatures of the first and second blocks.

The glass transition temperatures indicated are, unless otherwise mentioned, theoretical Tgs determined from the theoretical Tgs of the constituent monomers of each of the blocks, which may be found in a reference manual such as the Polymer Handbook, 4th edition (Brandrup, Immergut, Grulke), 1999, John Wiley, according to the following relationship, known as Fox's law:

$$\frac{1}{Tg} = {\sum }_{i} ( \frac{ϖ i}{Tgi} )$$

wi being the mass fraction of the monomer i in the block under consideration and Tgi being the glass transition temperature of the homopolymer of the monomer i (expressed in Kelvins).

The block polymer according to the invention thus comprises at least one block with a Tg of less than or equal to 20° C., and preferably at least one block with a Tg strictly greater than 20° C.

Preferably, one of the blocks has a Tg of between 20° C. (exclusive) and 160° C., especially between 40° C. and 120° C. and preferentially between 50° C. and 110° C.

Preferably, one of the blocks has a Tg of between −150° C. and 20° C. (inclusive), especially between −100° C. and 10° C. and preferentially between −50° C. and 0° C.

Preferably, the block having the highest Tg is in majority amount, by weight, relative to the other blocks.

Preferably, the block polymer according to the invention is formed exclusively from a first block and second block (it is then a diblock polymer), or exclusively from a first block, a second block and an intermediate segment that is preferably an intermediate block (which may thus be a triblock polymer).

When the block polymer does not comprise an intermediate block or segment (diblock), the block having the highest Tg may be present in the block polymer in an amount of from 50% to 90% by weight, especially 55% to 80% and better still from 60% to 75% by weight, relative to the total weight of the block polymer, and the second block may be present in an amount of from 10% to 50% by weight, especially 20% to 45% by weight and better still from 25% to 40% by weight relative to the total weight of the block polymer.

When the block polymer comprises an intermediate block or segment, the block having the highest Tg may be present in the block polymer in an amount of from 45% to 90% by weight, especially 50% to 80% and better still from 55% to 75% by weight relative to the total weight of the block polymer; the second block may be present in an amount of from 9% to 45% by weight, especially 10% to 40% by weight and better still from 20% to 35% by weight relative to the total weight of the block polymer; the intermediate segment or block may represent 1% to 10% by weight, especially 2% to 7% by weight and better still 3% to 5% by weight, relative to the total weight of the block polymer.

The first and/or second blocks may be formed from a homopolymer or a copolymer, which may be statistical, alternating or the like, preferably statistical.

The chemical nature and/or the amount of the constituent monomers of each of the blocks may obviously be chosen by a person skilled in the art, on the basis of his general knowledge, to obtain blocks that have the required Tgs.

An important characteristic of the polymers according to the invention is that the block with a Tg of less than or equal to 20° C. comprises 0.5% to 100% by weight, relative to the weight of this block, of at least one monomer of formula (I), or a mixture of such monomers:

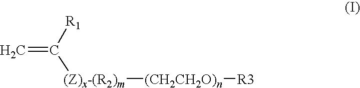

in which:
R1 is a hydrogen atom or a methyl radical;
Z is a divalent group chosen from —COO—, —CONH—, —CONCH₃—, —OCO—, —O—, —SO₂—, —CO—O—CO— and —CO—CH₂—CO—;
x is 0 or 1;
R2 is a saturated or unsaturated, optionally aromatic, linear, branched or cyclic carbon-based divalent radical of 1 to 30 carbon atoms, possibly comprising 1 to 18 heteroatoms chosen from O, N, S, F, Si and P;
m is 0 or 1;
n is an integer between 3 and 300 inclusive;
R3 is a hydrogen atom or a saturated or unsaturated, optionally aromatic, linear, branched or cyclic carbon-based radical of 1 to 30 carbon atoms, possibly comprising 1 to 20 heteroatoms chosen from O, N, S, F, Si and P.

Preferably, x=1 and Z represents COO or CONH, preferentially COO.

Preferably, the block with a Tg of less than or equal to 20° C. comprises 0.5, 1, 3, 6, 9, 15, 20, 25, 30, 35, 40, 45, 50, 55, 60, 65, 70, 75, 80, 85, 90, 95 or 100% by weight, relative to the weight of this block, of at least one monomer of formula (I), or a mixture of such monomers, including all values and subranges between stated values.

In the radical R2, the heteroatom(s), when it is (they are) present, may be intercalated in the chain of the radical R2, or alternatively the radical R2 may be substituted with one or more groups comprising them such as hydroxyl, amino (NH₂, NHR′ or NR′R″ with R′ and R″, which may be identical or different, representing a linear or branched C1-C22 alkyl, especially methyl or ethyl), —CF3, —CN, —SO3H or —COOH.

In particular, R2 may comprise a group —O—, —N(R)— or —CO— and their combination, and especially —O—CO—O—, —CO—O—, —N(R)CO—; —O—CO—NR—, —NR—CO—NR—, with R representing H or a linear or branched C1-C22 alkyl, optionally comprising 1 to 12 heteroatoms chosen from O, N, S, F, Cl, Br, Si and P.

R2 may especially be:
an alkylene radical containing 1 to 20 carbon atoms, such as methylene, ethylene, n-propylene, isopropylene, n-butylene, isobutylene, tert-butylene, pentylene, isopentylene, n-hexylene, isohexylene, heptylene, isoheptylene, n-octylene, isooctylene, nonylene, isononylene, decylene, isodecylene, n-dodecylene, isododecylene, tridecylene, n-tetradecylene, hexadecylene, n-octadecylene, docosanylene or arachinyene;
a substituted or unsubstituted cycloalkylene radical containing 5 to 10 carbon atoms, such as cyclopentylene, cyclohexylene, cycloheptylene, cyclooctylene, cyclononylene or cyclodecylene;
a phenylene radical —C₆H₄— (ortho, meta or para), optionally substituted with a C1-C12 alkyl radical optionally comprising 1 to 18 heteroatoms chosen from O, N, S, F, Si and P;
a benzylene radical —C₆H₄—CH₂— optionally substituted with a C1-C12 alkyl radical optionally comprising 1 to 18 heteroatoms chosen from O, N, S, F, Si and P;
a radical of formula —CH₂—O—CO—O—, CH₂—CH₂—O—CO—O—, —CH₂—CO—O—, —CH₂—CH₂—CO—O—, —CH₂—O—CO—NH—, —CH₂—CH₂—O—CO —NH—; —CH₂—NH—CO—NH—, —CH₂—CH₂—NH—CO—NH—; —CH₂—CHOH—, —CH₂—CH₂—CHOH—, —CH₂—CH₂—CH(NH₂)—, —CH₂—CH(NH₂)—, —CH₂—CH₂—CH(NHR′)—, —CH₂—CH(NHR′)—, —CH₂—CH₂—CH(NR′R″)—, —CH₂—CH(NR′R″)—, —CH₂—CH₂—CH₂—NR′—, —CH₂—CH₂—CH₂—O—; —CH₂—CH₂—CHR′—O— with R′ and R″ representing a linear or branched C1-C22 alkyl optionally comprising 1 to 12 heteroatoms chosen from O, N, S, F, Si and P;
or a mixture of these radicals.

Preferably, R2 may be:
an alkylene radical containing 1 to 20 carbon atoms, especially methylene, ethylene, n-propylene, n-butylene, n-hexylene, n-octylene, n-dodecylene or n-octadecylene;

a phenylene radical —C₆H₄— (ortho, meta or para), optionally substituted with a C1-C12 alkyl radical optionally comprising 1 to 18 heteroatoms chosen from O, N, S, F, Si and P; or
a benzylene radical —C₆H₄—CH₂— optionally substituted with a C1-C12 alkyl radical optionally comprising 1 to 18 heteroatoms chosen from O, N, S, F, Si and P.

Preferably, n is between 5 and 200 inclusive, better still between 6 and 120 inclusive, or even between 7 and 50 inclusive.

Preferably, R3 is a hydrogen atom; a phenyl radical optionally substituted with a C1-C12 alkyl radical optionally comprising 1 to 20 heteroatoms chosen from O, N, S, F, Si and P; a C1-C30, especially C1-C22 or even C2-C16 alkyl radical optionally comprising 1 to 18 heteroatoms chosen from O, N, S, F, Si and P; a C3-C12, especially C4-C8 or even C5-C6 cycloalkyl radical optionally comprising 1 to 18 heteroatoms chosen from O, N, S, F, Si and P.

Among the radicals R3, mention may be made of methyl, ethyl, propyl, benzyl, ethylhexyl, lauryl, stearyl, behenyl (—(CH₂)₂₁—CH₃), and also fluoroalkyl chains, for instance heptadecafluorooctyl sulfonyl amino ethyl CF₃—(CF₂)₇—SO₂—N(C₂H₅)—CH₂—CH₂; or alternatively —CH₂—CH₂—CN, succinimido, maleimido, mesityl, tosyl, triethoxysilane or phthalimide chains.

Preferentially, the monomers of formula (I) are such that:
x=1 and Z represents COO,
m=0,
n=6 to 120 inclusive,
R3 is chosen from a hydrogen atom; a phenyl radical optionally substituted with a C1-C12 alkyl radical; a C1-C30, especially C1-C22 or even C2-C16 alkyl radical.

Preferably, the monomers of formula (I) have a molecular weight of between 300 and 5000 g/mol.

Among the monomers of formula (I) that are particularly preferred, mention may be made of:
poly(ethylene glycol) (meth)acrylate in which R1 is H or methyl; Z is COO, x=1, m=0 and R3=H;
methyl-poly(ethylene glycol) (meth)acrylate, also known as methoxy-poly(ethylene glycol) (meth)acrylate, in which R1 is H or methyl, Z is COO, x=1, m=0 and R3=methyl;
alkyl-poly(ethylene glycol) (meth)acrylates in which R1 is H or methyl, Z is COO, x=1, m=0 and R3=alkyl;
phenyl-poly(ethylene glycol) (meth)acrylates, also known as poly(ethylene glycol) phenyl ether (meth)acrylate, in which R1 is H or methyl, Z is COO, x=1, m=0 and R3=phenyl.

Examples of commercial monomers are:
CD 350 (methoxy-poly(ethylene glycol 350) methacrylate) and CD 550 (methoxy-poly(ethylene glycol 550) methacrylate), sold by Sartomer Chemicals;
M90G (methoxy-poly(ethylene glycol (9 repeating units)) methacrylate) and M230G (methoxy-polyethylene glycol (23 repeating units) methacrylate) available from Shin-Nakamura Chemicals;
methoxy-poly(ethylene glycol) methacrylates of average molecular weights 300, 475 or 1100, available from Sigma-Aldrich;
methoxy-poly(ethylene glycol) acrylate of average molecular weight 426, available from Sigma-Aldrich;
methoxy-poly(ethylene glycol) methacrylates available from Laporte under the trade names: MPEG 350, MPEG 550, S10W or S20W, or from Cognis under the name Bisomer;
poly(ethylene glycol) monomethyl ether, mono(succinimidyl succinate) ester of average molecular weight 1900 or 5000, from Polysciences;
behenyl poly(ethylene glycol PEG-25) methacrylate available from Rhodia under the name Sipomer BEM;
poly(ethylene glycol) phenyl ether acrylates of average molecular weights 236, 280 or 324 available from Aldrich;
methoxy polyethylene glycol 5000 2-(vinylsulfonyl)ethyl ether commercially available from Fluka;
polyethylene glycol ethyl ether methacrylate available from Aldrich;
polyethylene glycol 8000, 4000, 2000 methacrylates from Monomer & Polymer Dajac Laboratories;
methoxy-poly(ethylene glycol) 2000 methacrylate Norsocryl 402 from Arkema;
methoxy-poly(ethylene glycol) 5000 methacrylate Norsocryl 405 from Arkema;
poly(ethylene glycol) methyl ether acrylate from Aldrich, Mn=454 g/mol, DP=8-9.

The monomers of formula (I) that are most particularly preferred are chosen from poly(ethylene glycol) (meth)acrylates and alkyl-poly(ethylene glycol) (meth)acrylates, more particularly methyl-poly(ethylene glycol) methacrylates.

Preferably, the monomer of formula (I) or the mixture of such monomers represents 0.7% to 95% by weight, especially 1% to 90% by weight, or even 5% to 87% by weight and better still 10% to 85% by weight relative to the total weight of monomers serving to form the block with a Tg of less than or equal to 20° C.

In one particular embodiment of the invention, the monomer of formula (I), alone or as a mixture, may also be present in another block of the polymer, especially in the block with a Tg strictly greater than 20° C.; in this case, it preferably represents 0.5% to 30% by weight, especially 1% to 25% by weight or even 2% to 20% by weight relative to the total weight of the block.

The other monomers that may be present in the polymer, thus constituting part of the block with a Tg of less than 20° C. and all or part of the other blocks, may be chosen, alone or as a mixture, from the following monomers:
(i) ethylenic hydrocarbons containing 2 to 10 carbons, such as ethylene, isoprene or butadiene;
(ii) the (meth)acrylates of formula CH₂═CHCOOR₃ or CH₂═C(CH₃)COOR₃ in which R₃ represents:
a saturated or unsaturated, linear or branched alkyl group containing 1 to 22 carbon atoms, especially to 20 or even 6 to 18 carbon atoms, which may optionally comprise, intercalated and/or as a substitution, one or more heteroatoms chosen from O, N, S and P and halogen atoms (Cl, Br, I and F);

R3 may especially be a methyl, ethyl, propyl, n-butyl, isobutyl, tert-butyl, hexyl, ethylhexyl, octyl, lauryl, isooctyl, isodecyl, dodecyl, behenyl or stearyl group; 2-ethylperfluorohexyl; or a C₁₋₄ hydroxyalkyl group such as 2-hydroxyethyl, 2-hydroxybutyl or 2-hydroxypropyl; or a (C₁₋₄)alkoxy(C₁₋₄)alkyl group such as methoxyethyl, ethoxyethyl or methoxypropyl;
a C₃-C₁₂ cycloalkyl group, such as an isobornyl, cyclohexyl or t-butylcyclohexyl group;
a C₃-C₂₀ aryl group such as a phenyl group;
a C₄-C₃₀ aralkyl group (C₁-C₈ alkyl group) such as 2-phenylethyl, t-butylbenzyl or benzyl;
a 4- to 12-membered heterocyclic group containing one or more heteroatoms chosen from O, N and S, the ring optionally being aromatic,
a heterocycloalkyl group (1 to 4 C alkyl), such as furfurylmethyl or tetrahydrofurfurylmethyl,

the cycloalkyl, aryl, aralkyl, heterocyclic or heterocycloalkyl groups possibly being optionally substituted with one or more substituents chosen from hydroxyl groups, halogen atoms and linear or branched C₁-C₄ alkyl groups, which may optionally comprise, intercalated and/or as a substitution, one or more heteroatoms chosen from O, N, S and P and halogen atoms (Cl, Br, I and F);
(iii) (meth)acrylamides of formula CH₂═CHCONR₆R₇ or CH₂═C(CH₃)CONR₆R₇ in which R₆ and R₇, which may be identical or different, represent:
a hydrogen atom; or
a saturated or unsaturated, linear or branched alkyl group containing 1 to 22 carbon atoms, especially to 20 or even 6 to 18 carbon atoms, which may optionally comprise, intercalated and/or as a substitution, one or more heteroatoms chosen from O, N, S and P and halogen atoms (Cl, Br, I and F);

R6 and/or R7 may especially be a methyl, ethyl, propyl, n-butyl, isobutyl, tert-butyl, hexyl, ethylhexyl, octyl, lauryl, isooctyl, isodecyl, dodecyl, behenyl or stearyl group; 2-ethylperfluorohexyl; or a C₁₋₄ hydroxyalkyl group such as 2-hydroxyethyl, 2-hydroxybutyl or 2-hydroxypropyl; or a (C₁₋₄)alkoxy(C₁₋₄)-alkyl group such as methoxyethyl, ethoxyethyl or methoxypropyl;
a C₃-C₁₂ cycloalkyl group, such as an isobornyl, cyclohexyl or t-butylcyclohexyl group;
a C₃-C₂₀ aryl group such as a phenyl group;

a C₄-C₃₀ aralkyl group (C₁-C₈ alkyl group) such as 2-phenylethyl, t-butylbenzyl or benzyl;
a 4- to 12-membered heterocyclic group containing one or more heteroatoms chosen from O, N and S, the ring optionally being aromatic,
a heterocycloalkyl group (C₁-C₄ alkyl), such as furfurylmethyl or tetrahydrofurfurylmethyl,

the cycloalkyl, aryl, aralkyl, heterocyclic or heterocycloalkyl groups possibly being optionally substituted with one or more substituents chosen from hydroxyl groups, halogen atoms and linear or branched C₁-C₄ alkyl groups, which may optionally comprise, intercalated and/or as a substitution, one or more heteroatoms chosen from O, N, S and P and halogen atoms (C1, Br, I and F).

Mention may be made especially of (meth) acrylamide, N-ethyl(meth)acrylamide, N-butyl(meth)acrylamide, N-t-butyl(meth)acrylamide, N-isopropyl(meth)acrylamide, N,N-dimethyl(meth)acrylamide, N,N-dibutyl(meth)acrylamide, N-octyl(meth)acrylamide, N-dodecyl(meth)acrylamide, N-undecyl(meth)acrylamide and N-2-hydroxypropyl(meth)acrylamide;
(iv) the vinyl compounds of formula CH₂═CH—R₉, CH₂═CH—CH₂—R₉ or CH₂═C(CH₃)—CH₂—R₉ in which R₉ is a hydroxyl group, halogen (Cl or F), NH₂, OR₁₄ in which R₁₄ represents a phenyl group or a C₁-C₁₂ alkyl group (the monomer is a vinyl or allyl ether); acetamide (NHCOCH₃); a group OCOR₁₅ in which R₁₅ represents a linear or branched alkyl group of 2 to 12 carbons (the monomer is a vinyl or allyl ester); or a group chosen from:
a linear or branched alkyl group of 1 to 22 carbon atoms, especially 4 to 20 or even 6 to 18 carbon atoms, which may optionally comprise, intercalated and/or as a substitution, one or more heteroatoms chosen from O, N, S and P and halogen atoms (Cl, Br, I and F);

a C₃-C₁₂ cycloalkyl group such as isobornyl or cyclohexyl,
a C₃-C₂₀ aryl group such as phenyl,
a C₄-C₃₀ aralkyl group (C₁-C₈ alkyl group) such as 2-phenylethyl or benzyl;
a 4- to 12-membered heterocyclic group containing one or more heteroatoms chosen from O, N and S, the ring optionally being aromatic;
a heterocycloalkyl group (C₁-C₄ alkyl) such as furfurylmethyl or tetrahydrofurfurylmethyl,

the cycloalkyl, aryl, aralkyl, heterocyclic or heterocycloalkyl groups possibly being optionally substituted with one or more substituents chosen from hydroxyl groups, halogen atoms and linear or branched C₁-C₄ alkyl groups, which may optionally comprise, intercalated and/or as a substitution, one or more heteroatoms chosen from O, N, S and P and halogen atoms (Cl, Br, I and F).

Mention may be made of vinylcyclohexane, styrene; vinyl acetate, vinyl propionate, vinyl butyrate, vinyl ethylhexanoate, vinyl neononanoate and vinyl neododecanoate; methyl vinyl ether, ethyl vinyl ether and isobutyl vinyl ether;
(v) ethylenically unsaturated monomers comprising at least one carboxylic, phosphoric or sulfonic acid or anhydride function, for instance acrylic acid, methacrylic acid, crotonic acid, maleic anhydride, itaconic acid, fumaric acid, maleic acid, acrylamidopropanesulfonic acid, vinylbenzoic acid or vinylphosphoric acid, and salts thereof;
(vi) ethylenically unsaturated monomers comprising at least one tertiary amine function, for instance 2-vinylpyridine, 4-vinylpyridine, dimethylaminoethyl methacrylate, diethylaminoethyl (meth)acrylate or dimethylaminopropyl(meth)acrylamide, and salts thereof.

The salts may be formed by neutralization of the anionic groups using a mineral base, such as LiOH, NaOH, KOH, Ca(OH)₂, NH₄OH or Zn(OH)₂; or with an organic base such as a primary, secondary or tertiary alkylamine, especially triethylamine or butylamine. This primary, secondary or tertiary alkylamine may comprise one or more nitrogen and/or oxygen atoms and may thus comprise, for example, one or more alcohol functions; mention may be made especially of 2-amino-2-methylpropanol, triethanolamine and 2-dimethylaminopropanol. Mention may also be made of lysine or 3-(dimethylamino)propylamine.

Mention may also be made of the salts of mineral acids, such as sulfuric acid, hydrochloric acid, hydrobromic acid, hydriodic acid or boric acid. Mention may also be made of the salts of organic acids, which may comprise one or more carboxylic, sulfonic or phosphonic acid groups. They may be linear, branched or cyclic aliphatic acids, or aromatic acids. These acids may also comprise one or more heteroatoms chosen from O and N, for example in the form of hydroxyl groups. Mention may be made especially of propionic acid, acetic acid, terephthalic acid, citric acid and tartaric acid.

Among the monomers whose homopolymers have a Tg of greater than 20° C., which may especially be present in the block with a Tg of greater than 20° C., mention may be made especially of:
the methacrylates of formula: CH₂═C(CH₃)—COOR₁

in which R₁ represents a linear or branched unsubstituted alkyl group containing from 1 to 4 carbon atoms, such as a methyl, ethyl, propyl or isobutyl group, or alternatively R₁ represents a C₄-C₁₂ cycloalkyl group, especially isobornyl;
the acrylates of formula: CH₂═CH—COOR₂

in which R₂ represents a tert-butyl group or a C₄-C₁₂ cycloalkyl group such as an isobornyl group;
the (meth)acrylamides of formula: CH₂═CR′—CO—NR₇R₈

with R′ representing H or CH₃, and R₇ and R₈, which may be identical or different, represent a hydrogen atom or a linear or branched C₁-C₁₂ alkyl group, such as an n-butyl, t-butyl, isopropyl, isohexyl, isooctyl or isononyl group; or alternatively R₇ represents H and R₈ represents a 1,1-dimethyl-3-oxobutyl group;
methacrylic acid and acrylic acid.

Mention may be made most particularly of methyl methacrylate, ethyl methacrylate, isobutyl methacrylate, tert-butyl (meth)acrylate, (meth)acrylic acid, isobornyl (meth)acrylate, N-butylacrylamide, N-t-butylacrylamide, N-isopropylacrylamide, N,N-dimethylacrylamide, N,N-dibutylacrylamide, n-butyl methacrylate, cyclodecyl acrylate, neopentyl acrylate and isodecylacrylamide, and mixtures thereof.

Among the monomers whose homopolymers have a Tg of less than 20° C., which may especially be present in the block with a Tg of less than 20° C., mention may be made especially of:
the acrylates of formula: CH₂═CH—COOR₃, in which R₂ represents a linear or branched, unsubstituted C₁-C₁₂ alkyl group, with the exception of a tert-butyl group, in which is (are) optionally intercalated one or more heteroatoms chosen from O, N and S;
the methacrylates of formula CH₂═C(CH₃)—COOR₄, in which R₄ represents a linear or branched, unsubstituted C₆-C₁₂ alkyl group, in which is (are) optionally intercalated one or more heteroatoms chosen from O, N and S;
vinyl esters of formula R₅—CO—O—CH═CH₂ in which R₅ represents a linear or branched C₄-C₁₂ alkyl group;
(C₄-C₁₂ alkyl) vinyl ethers, especially methyl vinyl ether and ethyl vinyl ether;

N—(C₄-C₁₂ alkyl)acrylamides, such as N-octylacrylamide;
and mixtures thereof.

Mention may be made most particularly of methyl acrylate, ethyl acrylate, isobutyl acrylate and 2-ethylhexyl (meth)acrylate, and mixtures thereof.

In one preferred embodiment, the polymer according to the invention comprises in at least one block, preferentially in each of the blocks, at least one monomer chosen from (meth)acrylic acid esters; optionally, it may also comprise at least one second monomer chosen from acrylic acid and methacrylic acid, or even a mixture thereof.

Preferentially, all the constituent monomers of the block polymer are chosen from (meth)acrylic acid esters and (meth)acrylic acid.

Thus, the monomers are more particularly chosen, alone or as a mixture, from:
C₁-C₂₂, especially C₄-C₂₀ or even C₆-C₁₈ alkyl (meth)acrylates, or C₃-C₁₂ cycloalkyl (meth)acrylates, and especially isobornyl (meth)acrylate, isobutyl (meth)acrylate, 2-ethylhexyl (meth)acrylate, dodecyl (meth)acrylate, stearyl (meth)acrylate, behenyl (meth)acrylate, methyl (meth)acrylate or tert-butyl (meth)acrylate;
(meth)acrylic acid,

and most particularly, alone or as a mixture, from:
C₁-C₄ alkyl or C₃-C₁₂ cycloalkyl methacrylates, and especially methyl methacrylate, isobornyl methacrylate or isobutyl methacrylate;
tert-butyl acrylate, methyl acrylate, isobutyl acrylate or isobornyl acrylate;
acrylic acid and methacrylic acid.

In one preferred embodiment, the block polymer comprises:
a first block obtained from at least one acrylate monomer of formula CH₂═CH—COOR₂ in which R₂ represents a C₄-C₁₂ cycloalkyl group and from at least one methacrylate monomer of formula CH₂═C(CH₃)—COOR′₂ in which R′₂ represents a C₄-C₁₂ cycloalkyl group;

the block preferably having a Tg strictly greater than 20° C., and
a second block obtained from at least one monomer of formula (I), from at least one additional monomer whose homopolymer has a Tg of less than or equal to 20° C., and optionally from at least one (meth)acrylic acid monomer; the block having a Tg of less than or equal to 20° C.

According to this embodiment, the first block may be obtained exclusively from the acrylate monomer and from the methacrylate monomer. The acrylate monomer and the methacrylate monomer are preferably in mass proportions of between 30/70 and 70/30, preferably between 40/50 and 50/40 and especially of about 50/50. The proportion of the first block advantageously ranges from 20% to 90%, better still from 30% to 80% and even better still from 60% to 80% by weight of the polymer. The first block is preferably obtained by polymerization of isobornyl methacrylate and isobornyl acrylate. The first block may also comprise (meth)acrylic acid, preferably acrylic acid; tert-butyl acrylate; a methacrylate of formula CH₂═C(CH₃)—COOR₁ in which R₁ represents a linear or branched, unsubstituted alkyl group containing from 1 to 4 carbon atoms, such as a methyl, ethyl, propyl or isobutyl group.

According to this embodiment, the second block is preferably obtained from the monomer of formula (I), acrylic acid and a monomer whose homopolymer has a Tg of less than or equal to 20° C., especially isobutyl acrylate.

Each of the first and/or second blocks may comprise, besides the monomers indicated above, one or more other monomers known as additional monomers, which are different from the main monomers mentioned previously. The additional monomers may represent 0.5% to 30% by weight relative to the weight of the polymer. Preferentially, in this embodiment, the polymer does not contain any additional monomer and is formed exclusively from the monomers indicated above.

The weight-average molar mass (Mw) of the block polymer according to the invention is preferably between 25 000 and 1 000 000, better still between 30 000 and 750 000, or even between 40 000 and 500 000 and preferentially between 50 000 and 250 000.

The weight-average (Mw) and number-average (Mn) molar masses are determined by gel permeation liquid chromatography (THF solvent, calibration curve established with linear polystyrene standards, refractometric and UV detector).

Preferably, the polydispersity index of the polymer according to the invention is greater than 2, for example ranging from 2 to 9, preferably greater than 2.5, for example ranging from 2.5 to 8 and better still ranging from 2.8 to 7. The polydispersity index Ip of the polymer is equal to the ratio of the weight-average mass Mw to the number-average mass Mn.

The block polymer may be obtained by radical solution polymerization according to the following preparation process:
part of the polymerization solvent may be introduced into a suitable reactor, and the system is heated until the appropriate temperature for the polymerization is reached (typically between 60 and 120° C.),
once this temperature has been reached, the constituent monomers of the first block may be added, in the presence of part of the polymerization initiator,
after a time T corresponding to a maximum degree of conversion preferably of 90%, the constituent monomers of the second block and the rest of the initiator may be introduced,
the mixture is left to react for a time T′ (especially ranging from 3 to 6 hours), after which the mixture is cooled to room temperature (25° C.) so as to obtain the polymer dissolved in the polymerization solvent.

The term “polymerization solvent” means a solvent, or a mixture of solvents, chosen especially from ethyl acetate, butyl acetate, C1-C6 alcohols such as isopropanol or ethanol, and aliphatic alkanes such as isododecane, and mixtures thereof. Preferably, the polymerization solvent is a mixture of butyl acetate and isopropanol or is isododecane.

Preferably, the block polymer is not water-soluble, i.e. it is not soluble in water or in a mixture of water and of linear or branched monoalcohols containing from 2 to 5 carbon atoms, such as ethanol, isopropanol or n-propanol, without pH modification, at an active material content of at least 1% by weight, at room temperature (25° C.)

Preferably, the block polymer, alone or as a mixture, may be present in a proportion of from 1% to 45% by weight in the composition according to the invention, especially 2% to 40% by weight or even 3% to 35% by weight relative to the total weight of the composition.

The cosmetic compositions according to the invention comprise, besides the polymers, a physiologically acceptable medium, especially a cosmetically or pharmaceutically acceptable medium, i.e. a medium that is compatible with keratin materials such as facial or bodily skin, the lips, the hair, the eyelashes, the eyebrows and the nails.

The composition according to the invention may advantageously comprise a liquid fatty phase, which may constitute a solvent medium for the polymers according to the invention, and which may comprise at least one compound chosen from volatile or non-volatile carbon-based, hydrocarbon-based, fluoro and/or silicone oils and/or solvents of mineral, animal, plant or synthetic origin, alone or as a mixture, provided that they form a stable homogeneous mixture and are compatible with the intended use.

For the purposes of the invention, the term “volatile” refers to any compound that is capable of evaporating on contact with keratin materials, or the lips, in less than one hour, at room temperature (25° C.) and atmospheric pressure (1 atm.). This volatile compound especially has a non-zero vapour pressure, at room temperature and atmospheric pressure, especially ranging from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg). In contrast, the term “non-volatile” refers to a compound that remains on keratin materials or the lips at room temperature and atmospheric pressure, for at least one hour, and which especially has a vapour pressure of less than 10⁻³ mmHg (0.13 Pa).

Preferably, the physiologically acceptable medium of the composition according to the invention may comprise, in a liquid fatty phase, at least one oil and/or one solvent, which may be chosen, alone or as a mixture, from:

1) esters of monocarboxylic acids with monoalcohols and polyalcohols; advantageously, the ester is a C12-C15 alkyl benzoate or corresponds to the following formula: R′₁—COO—R′₂ in which:

R′₁ represents an optionally substituted, linear or branched alkyl radical of 1 to 40 carbon atoms and preferably from 7 to 19 carbon atoms, optionally comprising one or more ethylenic double bonds, the hydrocarbon-based chain of which may be interrupted with one or more heteroatoms chosen from N and O and/or one or more carbonyl functions, and

R′₂ represents an optionally substituted, linear or branched alkyl radical of 1 to 40 carbon atoms, preferably from 3 to 30 carbon atoms and better still from 3 to 20 carbon atoms, optionally comprising one or more ethylenic double bonds, and the hydrocarbon-based chain of which may be interrupted with one or more heteroatoms chosen from N and O and/or one or more carbonyl functions.

The term “optionally substituted” means that R′₁ and/or R′₂ may bear one or more substituents chosen, for example, from groups comprising one or more heteroatoms chosen from 0 and/or N, such as amino, amine, alkoxy or hydroxyl.

Examples of groups R′₁ are those derived from fatty acids, preferably higher fatty acids, chosen from the group constituted by acetic acid, propionic acid, butyric acid, caproic acid, caprylic acid, pelargonic acid, capric acid, undecanoic acid, lauric acid, myristic acid, palmitic acid, stearic acid, isostearic acid, arachidic acid, behenic acid, oleic acid, linoleic acid, linolenic acid, oleostearic acid, arachidonic acid and erucic acid, and mixtures thereof.

Preferably, R′₁ is an unsubstituted branched alkyl group of 4 to 14 carbon atoms, preferably from 8 to 10 carbon atoms, and R′₂ is an unsubstituted branched alkyl group of 5 to 15 carbon atoms and preferably from 9 to 11 carbon atoms.

In particular, mention may preferably be made of C₈-C₄₈ esters, optionally incorporating in their hydrocarbon-based chain one or more heteroatoms chosen from N and O and/or one or more carbonyl functions; and more particularly purcellin oil (cetostearyl octanoate), isononyl isononanoate, isopropyl myristate, isopropyl palmitate, 2-ethylhexyl palmitate, 2-octyldodecyl stearate, 2-octyldodecyl erucate, isostearyl isostearate, C₁₂-C₁₅ alkyl benzoates, hexyl laurate or diisopropyl adipate; and heptanoates, octanoates, decanoates or ricinoleates of alcohols or of polyalcohols, for example of fatty alcohols, such as propylene glycol dioctanoate, and also isopropyl N-lauroyl sarcosinate (especially Eldew-205SL from Ajinomoto); hydroxylated esters, for instance isostearyl lactate or diisostearyl malate; and pentaerythritol esters; branched C₈-C₁₆ esters, especially isohexyl neopentanoate.

2) hydrocarbon-based plant oils with a high content of triglycerides, constituted of fatty acid esters of glycerol, the fatty acids of which may have varied chain lengths from C₄ to C₂₄, these chains possibly being linear or branched, and saturated or unsaturated; these oils are especially wheatgerm oil, corn oil, sunflower oil, shea oil, castor oil, sweet almond oil, macadamia oil, apricot oil, soybean oil, rapeseed oil, cottonseed oil, alfalfa oil, poppy seed oil, pumpkin oil, sesame seed oil, marrow oil, avocado oil, hazelnut oil, grapeseed oil, blackcurrant oil, evening primrose oil, millet oil, barley oil, quinoa oil, olive oil, rye oil, safflower oil, candlenut oil, passion flower oil, musk rose oil, jojoba oil, palm oil or beauty-leaf oil; or alternatively caprylic/capric acid triglycerides, such as those sold by the company Stéarineries Dubois or those sold under the names Miglyol 810®, 812® and 818® by the company Dynamit Nobel.

3) C6-C32 and especially C12-C26 alcohols, and especially monoalcohols, for instance oleyl alcohol, linoleyl alcohol, linolenyl alcohol, isostearyl alcohol, 2-hexyldecanol, 2-butyloctanol, 2-undecyl-pentadecanol and octyldodecanol.

4) linear or branched, volatile or non-volatile hydrocarbon-based oils, of synthetic or mineral origin, which may be chosen from hydrocarbon-based oils containing from 5 to 100 carbon atoms, and especially petroleum jelly, polydecenes, hydrogenated polyisobutenes such as Parleam, squalane and perhydrosqualene, and mixtures thereof.

Mention may be made more particularly of linear, branched and/or cyclic C5-C48 alkanes, and preferentially branched C8-C16 alkanes, for instance C8-C16 isoalkanes possibly of petroleum origin (also known as isoparaffins); especially decane, heptane, undecane, dodecane, tridecane and cyclohexane; and also isododecane, isodecane and isohexadecane, and mixtures thereof.

5) volatile or non-volatile silicone oils;

Volatile silicone oils that may be mentioned include volatile linear or cyclic silicone oils, especially those with a viscosity of less than 8 centistokes, and especially containing from 2 to 10 silicon atoms, these silicones optionally comprising alkyl or alkoxy groups containing from 1 to 22 carbon atoms; and in particular octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane and methylhexyldimethylsiloxane, and mixtures thereof.

The non-volatile silicone oils that may be used according to the invention may be polydimethylsiloxanes (PDMS), polydimethylsiloxanes comprising alkyl or alkoxy groups, which are pendent and/or at the end of a silicone chain, these groups each containing from 2 to 24 carbon atoms, phenyl silicones, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates.

In one preferred embodiment, the volatile oils, especially carbon-based oils, alone or as a mixture, are present in the composition in an amount of between 30% and 80% by weight, especially 35% to 75% by weight or even 40% to 70% by weight relative to the total weight of the composition.

The liquid fatty phase may also comprise additional oils and/or solvents, which may be chosen, alone or as a mixture, from:
fluoro oils such as perfluoropolyethers, perfluoroalkanes, for instance perfluorodecalin, perfluoroadamantanes, monoesters, diesters and triesters of perfluoroalkyl phosphates, and fluoro ester oils;
oils of animal origin;
C₆-C₄₀ and especially C₁₀-C₄₀ ethers; propylene glycol ethers that are liquid at room temperature, such as propylene glycol monomethyl ether, propylene glycol monomethyl ether acetate or dipropylene glycol mono-n-butyl ether;
C₈-C₃₂ fatty acids, for instance oleic acid, linoleic acid or linolenic acid, and mixtures thereof;
difunctional oils, comprising two functions chosen from ester and/or amide and containing from 6 to 30 carbon atoms, especially 8 to 28 carbon atoms and better still from 10 to 24 carbon atoms, and 4 heteroatoms chosen from O and N; the amide and ester functions preferably being in the chain;
ketones that are liquid at room temperature (25° C.) such as methyl ethyl ketone, methyl isobutyl ketone, diisobutyl ketone, isophorone, cyclohexanone or acetone;
aldehydes that are liquid at room temperature, such as benzaldehyde and acetaldehyde.

The liquid fatty phase may represent 5% to 90% by weight of the composition, especially from 10% to 75% by weight, in particular from 15% to 60% by weight or even from 25% to 55% by weight relative to the total weight of the composition.

The composition according to the invention may also comprise one or more physiologically acceptable organic solvents.

These solvents may generally be present in a content ranging from 0.1% to 90%, preferably from 0.5% to 85%, more preferably from 10% to 80% and better still from 30% to 50% by weight relative to the total weight of the composition.

Besides the hydrophilic organic solvents mentioned hereinabove, mention may be made especially of ketones that are liquid at room temperature, such as methyl ethyl ketone, methyl isobutyl ketone, diisobutyl ketone, isophorone, cyclohexanone and acetone; propylene glycol ethers that are liquid at room temperature, such as propylene glycol monomethyl ether, propylene glycol monomethyl ether acetate and dipropylene glycol mono-n-butyl ether; short-chain esters (containing from 3 to 8 carbon atoms in total) such as ethyl acetate, methyl acetate, propyl acetate, n-butyl acetate or isopentyl acetate; ethers that are liquid at 25° C., such as diethyl ether, dimethyl ether or dichlorodiethyl ether; alkanes that are liquid at 25° C., such as decane, heptane, dodecane, isododecane or cyclohexane; cyclic aromatic compounds that are liquid at 25° C., such as toluene and xylene; aldehydes that are liquid at 25° C., such as benzaldehyde and acetaldehyde, and mixtures thereof.

The composition may also comprise fatty substances that are solid at room temperature, such as waxes, pasty fatty substances and gums, and mixtures thereof. They may be of animal, plant, mineral or synthetic origin.

For the purposes of the present invention, the term “wax” means a lipophilic compound, which is solid at room temperature (25° C.), with a reversible solid/liquid change of state, having a melting point of greater than or equal to 25° C., which may be up to 120° C. By bringing the wax to the liquid state (melting), it is possible to make it miscible with the oils that may be present and to form a microscopically homogeneous mixture, but on returning the temperature of the mixture to room temperature, recrystallization of the wax in the oils of the mixture is obtained. The melting point of the wax may be measured using a differential scanning calorimeter (DSC), for example the calorimeter sold under the name DSC 30 by the company Mettler. The waxes may be hydrocarbon-based, fluoro and/or silicone waxes and may be of plant, mineral, animal and/or synthetic origin. In particular, the waxes have a melting point of greater than 30° C. and better still greater than 45° C. As waxes that may be used in the composition of the invention, mention may be made of beeswax, carnauba wax, candelilla wax, paraffin wax, microcrystalline waxes, ceresin or ozokerite; synthetic waxes, for instance polyethylene waxes or Fischer-Tropsch waxes, and silicone waxes, for instance alkyl or alkoxy dimethicones containing from 16 to 45 carbon atoms.

The gums are generally high molecular weight polydimethylsiloxanes (PDMS) or cellulose gums or polysaccharides, and the pasty fatty substances are generally hydrocarbon-based compounds, for instance lanolins and derivatives thereof or PDMSs.

The term “pasty fatty substance” means a viscous product containing a liquid fraction and a solid fraction. Fatty substances with a melting point ranging from 20 to 55° C. and/or a viscosity at 40° C. ranging from 0.1 to 40 Pa·s (1 to 400 poises), measured with a Contraves TV or Rheomat 80 viscometer, are especially intended. A person skilled in the art can select the spindle for measuring the viscosity from the spindles MS-r3 and MS-r4, on the basis of his general knowledge, so as to be able to measure the viscosity of the pasty compound tested. The melting point values correspond, according to the invention, to the melting peak measured by the differential scanning colorimetry method, with a temperature rise of 5 or 10° C./minute. Preferably, these fatty substances are hydrocarbon-based compounds (mainly containing carbon and hydrogen atoms and possibly ester groups), optionally of polymeric type; they may also be chosen from silicone and/or fluoro compounds; they may also be in the form of a mixture of hydrocarbon-based and/or silicone and/or fluoro compounds. In the case of a mixture of different pasty fatty substances, hydrocarbon-based pasty compounds are preferably used in majority proportion. Among the pasty compounds that may be used in the composition according to the invention, mention may be made of lanolins and lanolin derivatives, for instance acetylated lanolins or oxypropylenated lanolins or isopropyl lanolate; esters of fatty acids or of fatty alcohols, especially those containing 20 to carbon atoms, for instance triisostearyl or cetyl citrate; arachidyl propionate; polyvinyl laurate; cholesterol esters, for instance triglycerides of plant origin such as hydrogenated plant oils, viscous polyesters, for instance poly(12-hydroxystearic acid), and mixtures thereof. Hydrogenated castor oil derivatives may be used as triglycerides of plant origin. Mention may also be made of silicone pasty fatty substances such as polydimethylsiloxanes (PDMS) containing pendent chains of the alkyl or alkoxy type containing from 8 to 24 carbon atoms, for instance stearyl dimethicones.

The nature and amount of the solid substances depend on the mechanical properties and textures sought. As a guide, the composition may contain from 0.1% to 50% by weight and better still from 1% to 30% by weight of waxes relative to the total weight of the composition.

The composition may also comprise a hydrophilic medium comprising water or a mixture of water and of one or more hydrophilic organic solvents, for instance alcohols and especially linear or branched lower monoalcohols containing from 2 to 5 carbon atoms, for instance ethanol, isopropanol or n-propanol; polyols, for instance glycerol, diglycerol, propylene glycol, sorbitol or pentylene glycol; polyethylene glycols, or alternatively hydrophilic C₂ ethers and C₂-C₄ aldehydes. The water or the mixture of water and of hydrophilic organic solvents may be present in the composition according to the invention in a content of from 10% to 80% by weight relative to the total weight of the composition. The composition may also be anhydrous.

The composition according to the invention may also comprise one or more dyestuffs chosen from pulverulent compounds, for instance pigments, fillers, nacres and glitter flakes, and/or liposoluble or water-soluble dyes.

The dyestuffs, especially pulverulent dyestuffs, may be present in the composition in a content of from 0.01% to 50% by weight, preferably from 0.1% to 40% by weight or even from 1% to 30% by weight relative to the weight of the composition.

The term “pigments” should be understood as meaning white or coloured, mineral or organic particles of any shape, which are insoluble in the physiological medium, and which are intended to colour the composition.

The term “nacres” should be understood as meaning iridescent particles of any shape, produced especially by certain molluscs in their shell, or alternatively synthesized.

The pigments may be white or coloured, mineral and/or organic, and interference or non-interference pigments. Among the mineral pigments that may be mentioned are titanium dioxide, optionally surface-treated, zirconium oxide or cerium oxide, and also zinc oxide, iron oxide or chromium oxide, manganese violet, ultramarine blue, chromium hydrate and ferric blue. Among the organic pigments that may be mentioned are carbon black, pigments of D&C type and lakes based on cochineal carmine or on barium, strontium, calcium or aluminium.

The nacreous pigments may be chosen from white nacreous pigments such as mica coated with titanium or with bismuth oxychloride, coloured nacreous pigments such as titanium mica coated with iron oxides, titanium mica coated especially with ferric blue or with chromium oxide, titanium mica coated with an organic pigment of the abovementioned type and also nacreous pigments based on bismuth oxychloride.

The fillers may be mineral or organic, and lamellar or spherical. Mention may be made of talc, mica, silica, kaolin, Nylon powders, poly-β-alanine powders and polyethylene powders, Teflon, lauroyllysine, starch, boron nitride, tetrafluoroethylene polymer powders, hollow microspheres such as Expancel (Nobel Industrie), Polytrap (Dow Corning) and silicone resin microbeads (for example Tospearls from Toshiba), and silicone resin microbeads (for example Tospearls from Toshiba), precipitated calcium carbonate, magnesium carbonate, magnesium hydrogen carbonate, hydroxyapatite, hollow silica microspheres (Silica Beads from Maprecos), glass or ceramic microcapsules, and metal soaps derived from organic carboxylic acids containing from 8 to 22 carbon atoms and preferably from 12 to 18 carbon atoms, for example zinc, magnesium or lithium stearate, zinc laurate or magnesium myristate.

The liposoluble dyes are, for example, Sudan red, DC Red 17, DC Green 6, β-carotene, soybean oil, Sudan brown, DC Yellow 11, DC Violet 2, DC Orange 5 or quinoline yellow. They may represent 0.01% to 20% and better still from 0.1% to 6% of the weight of the composition.

The water-soluble dyes are, for example, beetroot juice or methylene blue, and may represent 0.01% to 6% of the total weight of the composition.

The composition according to the invention may also comprise one or more fillers, especially in a content ranging from 0.01% to 50% by weight and preferably ranging from 0.02% to 30% by weight relative to the total weight of the composition. The term “fillers” should be understood as meaning colourless or white, mineral or synthetic, lamellar or non-lamellar particles, which are intended to give the composition body or rigidity, and/or to give the makeup result softness, a matt effect and uniformity. The fillers may be mineral or organic and of any shape: platelet-shaped, spherical or oblong. Mention may be made of talc, mica, silica, kaolin, polyamide) (Nylon®) powders, poly-β-alanine powders and polyethylene powders, powders of tetrafluoroethylene polymers) (Teflon®), lauroyllysine, starch, boron nitride, hollow polymer microspheres such as Expancel® (Nobel Industrie), acrylic acid copolymers (Polytrap® from the company Dow Corning) and silicone resin microbeads (for example Tospearls® from Toshiba), and silicone resin microbeads (for example Tospearls from Toshiba), elastomeric polyorganosiloxane particles, precipitated calcium carbonate, magnesium carbonate, magnesium hydrogen carbonate, hydroxyapatite, hollow silica microspheres (Silica Beads® from Maprecos), glass or ceramic microcapsules, and metal soaps derived from organic carboxylic acids containing from 8 to 22 carbon atoms and preferably from 12 to 18 carbon atoms, for example zinc, magnesium or lithium stearate, zinc laurate or magnesium myristate.

The composition may also comprise an additional polymer such as a film-forming polymer. According to the present invention, the term “film-forming polymer” means a polymer that is capable of forming, by itself or in the presence of a film-forming auxiliary, a continuous film that adheres to a support, especially to keratin materials. Among the film-forming polymers that may be used in the composition of the present invention, mention may be made of synthetic polymers, of free-radical type or of polycondensate type, polymers of natural origin, and mixtures thereof, in particular acrylic polymers, polyurethanes, polyesters, polyamides, polyureas, and cellulose-based polymers, for instance nitrocellulose.

The composition according to the invention may also comprise ingredients commonly used in cosmetics, such as vitamins, thickeners, gelling agents, trace elements, softeners, sequestrants, fragrances, acidifying or basifying agents, preserving agents, sunscreens, surfactants, antioxidants, hair-loss counteractants, antidandruff agents, propellants, ceramides or film-forming auxiliaries, or mixtures thereof.

Needless to say, a person skilled in the art will take care to select this or these optional additional compound(s), and/or the amount thereof, such that the advantageous properties of the composition according to the invention are not, or are not substantially, adversely affected by the envisaged addition.

The composition according to the invention may be in the form of a suspension, a dispersion especially of oil in water by means of vesicles; an optionally thickened or even gelled aqueous or oily solution; an oil-in-water, water-in-oil or multiple emulsion; a gel or a mousse; an oily or emulsified gel; a dispersion of vesicles, especially lipid vesicles; a two-phase or multi-phase lotion; a spray; a loose, compact or cast powder; an anhydrous paste. This composition may have the appearance of a lotion, a cream, a pomade, a soft paste, an ointment, a mousse, a cast or moulded solid, especially in stick or dish form, or alternatively a compacted solid.

A person skilled in the art can select the appropriate galenical form, and also the method for preparing it, on the basis of his general knowledge, taking into account firstly the nature of the constituents used, especially their solubility in the support, and secondly the intended use of the composition.

The cosmetic composition according to the invention may be in the form of a product for caring for and/or making up bodily or facial skin, the lips, the nails, the eyelashes, the eyebrows and/or the hair, an antisun or self-tanning product, or a haircare product for caring for, treating, shaping, making up or colouring the hair.

It may thus be in the form of a makeup composition, especially a complexion product such as a foundation, a makeup rouge or an eyeshadow; a lip product such as a lipstick, a lip gloss or a lipcare product; a concealer product; a blusher, a mascara or an eyeliner; a makeup product for the eyebrows, a lip pencil or an eye pencil; a nail product such as a nail varnish or a nailcare product; a body makeup product; a hair makeup product (hair lacquer or mascara).

It may also be in the form of a composition for protecting or caring for the skin of the face, the neck, the hands or the body, especially an anti-wrinkle composition or a moisturizing or medicated composition; an antisun or artificial-tanning (self-tanning) composition.

It may also be in the form of a haircare product, especially for colouring, holding the hairstyle or shaping the hair, for caring for, treating or cleansing the hair, such as shampoos, hair conditioners, hair-setting gels or lotions, blow-drying lotions, or fixing and styling compositions such as lacquers or sprays.

Preferably, the cosmetic composition according to the invention is in the form of a makeup product, especially a lipstick, a lip gloss, a mascara, a nail varnish or a foundation, or a care product such as a facial care cream or an antisun product.

A subject of the invention is also a cosmetic treatment process, especially for making up or caring for keratin materials such as bodily or facial skin, the lips, the nails, the hair, the eyebrows and/or the eyelashes, comprising the application to the materials of a cosmetic composition as defined previously.

This process makes it possible especially to make up the skin, the eyelashes, the nails, the hair and/or the lips.

The invention is illustrated in greater detail in the examples that follow.

Gloss Measured with a Glossmeter on a Dry Deposit of Polymer

The gloss may be measured using a glossmeter in a conventional manner via the following method.

A coat 50 μm thick of polymer to be tested, as a solution at 50% in isododecane, is spread using a spreader onto a contrast card of Leneta brand and of reference Form 1A Penopac. The coat covers at least the black background of the card. The deposit is left to dry for 24 hours at a temperature of 25° C., and the gloss at 20° is then measured on the black background using a glossmeter of Dr Lange brand, Ref03. The gloss at 60° is also measured as previously.

Resistance to Olive Oil by Measuring the Tack Aspect

This is determined with a drop of olive oil placed on a film of dry polymer.

A polymer film is prepared from a solution containing 20% by weight of block polymer in isododecane; 0.5 ml is spread onto a 2.5×7.5 cm glass plate and left to dry at room temperature (25° C.) for 24 hours. Next, 1 ml of olive oil is spread onto the film.

After the desired time (2 hours), the excess oil is wiped from the film and the tack aspect is evaluated by feel.

A grade + is given when the tack is perceptible after a short pressure (about 1 second) with a finger.

A grade − is given when no tack is detected, after a short pressure with a finger.

The tack reflects the sensitivity of the polymer to olive oil. The greater the tack, the more sensitive the polymer to the oil and thus the more easily the deposit will be impaired, for example at mealtimes (in the presence of food oil) or by sebum. This results in poorer staying power of the polymer on the skin. This also results in a decrease in the comfort: the tackier the film, the less comfortable the composition is to wear.

Determination of the Brittleness

A coat 50 μm thick of polymer as a solution at 50% in isododecane is spread using a spreader onto a contrast card of Leneta brand and of reference Form 1A Penopac. The coat covers at least the black background of the card. The deposit is left to dry for 24 hours at a temperature of 25° C. The card is curved and the formation of cracks is observed.

A grade +++ means that the film is very brittle, pieces detach from the card, and the film cracks even without the card being curved.

A grade + means that the film is sparingly brittle, only a few cracks appear, and the film remains cohesive.

Method for Measuring the Viscosity of the Polymers

The viscosity at 25° C. of the block polymer is measured using a cylindrical viscometer of Brookfield DV-I+ type.

General Process for Synthesizing the Polymers

1/Synthesis of the comparative poly(isobornyl acrylate/isobornyl methacrylate/isobutyl acrylate/acrylic acid)

300 g of isododecane are placed in a 1 litre reactor, and the temperature is then raised so as to pass from room temperature (25° C.) to 90° C. in 1 hour. 105 g of isobornyl methacrylate, 105 g of isobornyl acrylate and 1.8 g of 2,5-bis(2-ethylhexanoylperoxy)-2,5-dimethylhexane (Trigonox® 141 from Akzo Nobel) are added, at 90° C. and over 1 hour 30 minutes.

75 g of isobutyl acrylate, 15 g of acrylic acid and 1.2 g of 2,5-bis(2-ethylhexanoylperoxy)-2,5-dimethylhexane are then added to the preceding mixture, still at 90° C. and over 30 minutes. The mixture is maintained at 90° C. for 3 hours, and is then cooled.

A solution with a polymer solids content of 50% in isododecane is obtained. The polymer comprises a poly(isobornyl acrylate/isobornyl methacrylate) first block with a Tg of 128° C., a poly(isobutyl acrylate/acrylic acid) second block with a Tg of −9° C. and an intermediate block, which is an isobornyl acrylate/isobornyl methacrylate/isobutyl acrylate/acrylic acid statistical polymer. These are theoretical Tg values calculated by means of Fox's law.

The solution containing 50% by weight of polymer in isododecane has a viscosity of 30 000 cps at 25° C.

2/Synthesis of the Polymers According to the Invention

The amounts of monomers are adapted for the syntheses of the polymers according to the invention, which are performed according to the preceding procedure.

### Examples 1 to 6

According to the general procedure described above, the following polymers are prepared (weight %):

| | | | Block of Tg ≦ 20° C. | | | |
|---|---|---|---|---|---|---|
| | Block of Tg > 20° C. | | | A- | | |
| | Isobornyl | Isobornyl | Isobutyl | crylic | MPEG | MPEG |
| | acrylate | methacrylate | acrylate | acid | 350* | 550** |
| Compar- | 35 | 35 | 25 | 5 | | |
| ative | | | | | | |
| Example 1 | 35 | 35 | 20 | 5 | 5 | |
| Example 2 | 35 | 35 | 20 | | 10 | |
| Example 3 | 35 | 35 | 15 | 5 | 10 | |
| Example 4 | 35 | 35 | 20 | 5 | | 5 |
| Example 5 | 35 | 35 | 10 | 5 | | 15 |
| Example 6 | 35 | 35 | | 5 | | 25 |

*MPEG 350: methoxypolyethylene glycol methacrylate of molar mass 350 g/mol (Bisomer MPEG 350 from Cognis)
**MPEG 550: methoxypolyethylene glycol methacrylate of molar mass 550 g/mol (Bisomer MPEG 550 from Cognis)

### Example 7

The gloss, the tack and the brittleness of the above polymers are determined, by comparison with the comparative compound.

| | Comparative | Example 1 | Example 2 | Example 3 |
|---|---|---|---|---|
| Viscosity (25° C.) | 30 000 cps | 7400 cps | 7800 cps | 3750 cps |
| of the solution | | | | |
| Film aspect | Glossy | Glossy | Glossy | Glossy |
| Gloss | 20° = 79.6 | 20° = 75.5 | 20° = 72.4 | 20° = 75.7 |
| | 60° = 88.7 | 60° = 88.4 | 60° = 87 | 60° = 88.3 |
| Tack (sensitivity | 2 hours: + | 2 hours: − | 2 hours: − | 2 hours: − |
| to olive oil) | | | | |
| Brittleness | +++ | ++ | + | ++ |

| | Example 4 | Example 5 | Example 6 |
|---|---|---|---|
| Viscosity (25° C.) | 15 600 cps | 3580 cps | 3100 cps |
| of the solution | | | |
| Film aspect | Glossy | Glossy | Glossy |
| Gloss | 20° = 77.3 | 20° = 80.5 | 20° = 71.2 |
| | 60° = 89.1 | 60° = 89.7 | 60° = 88.6 |
| Tack (sensitivity | 2 hours: − | 2 hours: − | 2 hours: − |
| to olive oil) | | | |
| Brittleness | ++ | ++ | ++ |

It is thus found that the films according to the invention are less tacky and less brittle than the films obtained with the comparative polymer.

Moreover, the presence of MPEG makes it possible to obtain polymers whose viscosity is lower, in comparison with the polymers of the prior art; this can facilitate the use of these polymers, especially the ease of handling and of incorporation into a composition.

### Example 8

Starting with a solution at 50% in isododecane, a film 50 μm thick (“wet” thickness, i.e. thickness of the solution as deposited) is produced on a Leneta card. A drop of water is spread onto each film and is left in contact for 24 hours. After wiping off the drop of water, the surface in contact is evaluated by feel.

For the films obtained with the solutions of polymers according to the invention, the surface is slippery and flexible (non-brittle). For the films obtained with the solution of comparative polymer, the surface is brittle and non-slippery.

### Example 9

### Mascara Composition

A mascara having the composition below is prepared:

| | | |
|---|---|---|
| Waxes (beeswax, paraffin wax, carnauba wax) | 17 | g |
| Modified hectorite (Bentone ® 38V) | 5.3 | g |
| Propylene carbonate | 1.7 | g |
| Pigments | 5 | g |
| Solution of polymer of Example 1 | 20 | g (i.e. |
| | | 10 g DM*) |
| Isododecane | qs 100 | g |

*DM: dry matter

The mascara, after application to the eyelashes, is judged to be very satisfactory.

A mascara is prepared in a similar manner with the polymers of Examples 2 to 6.

### Example 10

### Lipstick

The lipstick composition below (weight %) is prepared:

| | |
|---|---|
| Polyethylene wax | 15% |
| Solution of polymer of Example 1 | 20% |
| | (i.e. 10% DM) |
| Hydrogenated polyisobutylene | 26% |
| (Parleam from Nippon Oil Fats) | |
| Pigments | 8.6% |
| Isododecane | qs 100% |

The composition obtained after application to the lips has good cosmetic properties.

A lipstick is prepared in a similar manner with the polymers of Examples 2 to 6.

### Example 11

### Foundation

A foundation comprising the compounds below is prepared:

| Phase A | Cetyl dimethicone copolyol | 3 | g |
|---|---|---|---|
| | (Abil EM 90 from the company | | |
| | Goldschmidt) | | |
| | Isostearyl diglyceryl succinate | 0.6 | g |
| | (Imwitor 780K from the company Condea) | | |
| | Isododecane | 18.5 | g |
| | Pigments | 10 | g |
| | Solution of polymer of Example 1 | 16 | g (i.e. |
| | | | 8 g DM) |
| | Polyamide powder (Nylon-12) | 8 | g |
| Phase B | Water | qs 100 | g |
| | Magnesium sulfate | 0.7 | g |
| | Preserving agent | qs | |
| Phase C | Water | 2 | g |

| | |
|---|---|
| Preserving agent | qs |

The composition obtained has good cosmetic properties.

A foundation is prepared in a similar manner with the polymers of Examples 2 to 6.

### Example 12

### Compact Powder

A compacted powder having the composition below is prepared:

Composition A:

| | | |
|---|---|---|
| Talc | 30 | g |
| Bismuth oxychloride | 10 | g |
| Zinc stearate | 4 | g |
| Nylon powder | 20 | g |
| Solution of polymer of Example 1 | 5 | g (i.e. 2.5 g DM) |

Composition B:

| | | |
|---|---|---|
| Iron oxides | 2 | g |
| Liquid petroleum jelly | 6 | g |

The powder is obtained in the following manner: composition A is ground in a mill of Kenwood type for about 5 minutes at low speed, composition B is added and the mixture is ground for about 2 minutes at the same speed, and then for 3 minutes at a higher speed. The preparation is then screened through a 0.16 mm screen, and this mixture is then compacted in dishes.

A compacted powder that has good cosmetic properties is obtained.

A powder is prepared in a similar manner with the polymers of Examples 2 to 6.

### Example 13

### Facial Gel

The composition below is prepared:

| | | |
|---|---|---|
| petroleum jelly (wax) | 5 | g |
| modified hectorite (clay) | 0.15 | g |
| ozokerite (wax) | 5 | g |
| oxyethylenated (40 EO) sorbitan | 5 | g |
| heptaoleate | | |
| solution of polymer of Example 1 | 50 | g (i.e. 25 g DM) |
| isopropyl palmitate | qs 100 | g |

A gel that has good cosmetic properties is obtained.

A facial gel is prepared in a similar manner with the polymers of Examples 2 to 6.

### Example 14

### Care Oil

The composition below is prepared:

| | | |
|---|---|---|
| solution of polymer of Example 1 | 50 | g (i.e. 25 g DM) |
| soybean oil | 15 | g |
| jojoba oil | qs 100 | g |

A care oil that can be applied to the body or the face is obtained.

A care oil is prepared in a similar manner with the polymers of Examples 2 to 6.

### Example 15

### Lip Gloss

A gloss having the composition below is prepared:

| | |
|---|---|
| polybutene | 34% |
| isononyl isononanoate | 4% |
| octyldodecanol | 10% |
| silica (Aerosil R972) | 5% |
| solution of polymer of Example 1 | 28% |
| | (i.e. 14% DM) |
| tridecyl trimellitate | qs 100 g |

A gloss is prepared in a similar manner with the polymers of Examples 2 to 6.

The above written description of the invention provides a manner and process of making and using it such that any person skilled in this art is enabled to make and use the same, this enablement being provided in particular for the subject matter of the appended claims, which make up a part of the original description.

As used herein, the words “a” and “an” and the like carry the meaning of “one or more.”

The phrases “selected from the group consisting of,” “chosen from,” and the like include mixtures of the specified materials. Terms such as “contain(s)” and the like are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A block polymer comprising at least one first block and at least one second block, which are mutually incompatible, in which one of the blocks has a glass transition temperature (Tg) of less than or equal to 20° C. and comprises 0.5% to 100% by weight, relative to the weight of the block, of at least one monomer of formula (I): 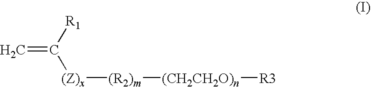
in which:
R1 is a hydrogen atom or a methyl radical;
Z is a divalent group chosen from —COO—, —CONH—, —CONCH₃—, —OCO—, —O—, —SO₂—, —CO—O—CO— and —CO—CH₂—CO—;
x is 0 or 1;
R2 is a saturated or unsaturated, optionally aromatic, linear, branched or cyclic carbon-based divalent radical of 1 to 30 carbon atoms, optionally comprising 1 to 18 heteroatoms chosen from O, N, S, F, Si and P;
m is 0 or 1;
n is an integer between 3 and 300 inclusive;
R3 is a hydrogen atom or a saturated or unsaturated, optionally aromatic, linear, branched or cyclic carbon-based radical of 1 to 30 carbon atoms, optionally comprising 1 to 20 heteroatoms chosen from O, N, S, F, Si and P.

2. The block polymer according to claim 1, in which the first and second blocks are connected together via an intermediate segment comprising at least one constituent monomer of the first block and at least one constituent monomer of the second block.

3. The block polymer according to claim 1, comprising at least one block with a Tg greater than 20° C.

4. The block polymer according to claim 1, in which, in formula (I):
x=1 and Z represents COO or CONH; and/or
R2 represents an alkylene radical containing 1 to 20 carbon atoms; a phenylene radical —C₆H₄— (ortho, meta or para), optionally substituted with a C1-C12 alkyl radical optionally comprising 1 to 18 heteroatoms chosen from O, N, S, F, Si and P; or a benzylene radical —C₆H₄—CH₂— optionally substituted with a C1-C12 alkyl radical optionally comprising 1 to 18 heteroatoms chosen from O, N, S, F, Si and P; and/or
n is between 5 and 200 inclusive; and/or
R3 is a hydrogen atom; a phenyl radical optionally substituted with a C1-C12 alkyl radical optionally comprising 1 to 20 heteroatoms chosen from O, N, S, F, Si and P; a C1-C30 alkyl radical optionally comprising 1 to 18 heteroatoms chosen from O, N, S, F, Si and P; a C3-C12 cycloalkyl radical optionally comprising 1 to 18 heteroatoms chosen from O, N, S, F, Si and P.

5. The block polymer according to claim 1, in which the monomers of formula (I) are such that:
x=1 and Z represents COO, and
m=0, and
n=6 to 120 inclusive, and
R3 is chosen from a hydrogen atom; a phenyl radical optionally substituted with a C1-C12 alkyl radical; and a C1-C30 alkyl radical.

6. The block polymer according to claim 1, in which the monomer of formula (I) is chosen from:
poly(ethylene glycol) (meth)acrylate in which R1 is H or methyl; Z is COO, x=1, m=0 and R3=H;
methyl-poly(ethylene glycol) (meth)acrylate in which R1 is H or methyl, Z is COO, x=1, m=0 and R3=methyl;
alkyl-poly(ethylene glycol) (meth)acrylates in which R1 is H or methyl, Z is COO, x=1, m=0 and R3=alkyl;
phenyl-poly(ethylene glycol) (meth)Acrylates in which R1 is H or methyl, Z is COO, x=1, m=0 and R3=phenyl.

7. The block polymer according to claim 1, in which the monomer of formula (I) or the mixture of such monomers represents 0.7% to 95% by weight relative to the total weight of monomers serving to form the block with a Tg of less than or equal to 20° C.

8. The block polymer according to claim 1, having a weight-average molar mass (Mw) of between 25 000 and 1 000 000.

9. The block polymer according to claim 1, having a polydispersity index of 2 to 9.

10. A cosmetic composition comprising, in a cosmetically acceptable medium, at least one block polymer as defined in claim 1.

11. The composition according to claim 10, in which the block polymer, alone or as a mixture, is present in a proportion of from 1% to 45% by weight relative to the total weight of the composition.

12. The composition according to claim 10, in which the cosmetically acceptable medium comprises at least one ingredient chosen from: volatile or non-volatile, carbon-based, hydrocarbon-based, fluoro and/or silicone oils and/or solvents of mineral, animal, plant or synthetic origin; fatty substances that are solid at room temperature, such as waxes, pasty fatty substances and gums; water; hydrophilic organic solvents; dyestuffs; polymers; vitamins, thickeners, gelling agents, trace elements, softeners, sequestrants, fragrances, acidifying or basifying agents, preserving agents, sunscreens, surfactants, antioxidants, hair-loss counteractants, antidandruff agents, propellants, ceramides, film-forming auxiliaries, and mixtures thereof.

13. The composition according to claim 10, which is in the form of a care and/or makeup product for bodily or facial skin, the lips, the nails, the eyelashes, the eyebrows and/or the hair, an antisun or self-tanning product, or a haircare product for caring for, treating, shaping, making up or colouring the hair.

14. The composition according to claim 10, which is in the form of a makeup product or a care product.

15. A process for treating a keratin material, comprising the application to the material of a cosmetic composition as defined in claim 10.

16. A process for making up the lips, comprising the application to the lips of a cosmetic composition as defined in claim 10.
